(19) United States
(12) Patent Application Publication
(10) Pub. No.: US 2024/0366511 A1
(43) Pub. Date: Nov. 7, 2024

(54) ORAL SOLID DOSE FORMULATIONS

(71) Applicant: GLAXOSMITHKLINE INTELLECTUAL PROPERTY DEVELOPMENT LIMITED, Stevenage (GB)

(72) Inventors: Chandan BHUGRA, Beijing (CN); Shavari BORKAR, Collegeville, PA (US); Yoon OH, Collegeville, PA (US); Rennan PAN, Collegeville, PA (US)

(21) Appl. No.: 18/262,938

(22) PCT Filed: Feb. 3, 2022
(86) PCT No.: PCT/EP2022/052564

§ 371 Date: Jul. 26, 2023

Related U.S. Application Data
(60) Provisional application No. 63/146,106, filed on Feb. 5, 2021.

(51) Int. Cl. A61K 9/20; A61K 31/4985

(52) U.S. Cl. CPC A61K 9/2054; A61K 9/2009; A61K 31/4985

(57) ABSTRACT

Disclosed are novel pharmaceutical compositions comprising gepotidacin, such as gepotidacin mesylate dihydrate, gepotidacin mesylate anhydrate or gepotidacin anhydrate. The present disclosure also provides methods for making the pharmaceutical composition comprising gepotidacin, and methods of treating bacterial infections using such pharmaceutical composition.

### STATEMENT REGARDING FEDERALLY SPONSORED RESEARCH

This invention was made with the US government support under Agreement No.: HDTRA1-07-9-0002 awarded by the Defense Threat Reduction Agency. The US government may have certain rights in the invention.

### BACKGROUND OF THE INVENTION

(2R)-2-({4-[(3,4-Dihydro-2H-pyrano[2,3-c]pyridin-6-ylmethyl)amino]-1-piperidinyl}methyl)-1,2-dihydro-3H,8H-2a,5,8a- triazaacenaphthylene-3,8-dione (hereinafter “gepotidacin”) selectively inhibits bacterial DNA gyrase and topoisomerase IV by a unique mechanism, which is not utilized by any currently approved human therapeutic agent.

International Patent Application Publication No. WO 2008/128942 describes a series of compounds which can be used as antibacterial agents, including gepotidacin. Mono-HCl salt of gepotidacin was prepared in Example 39 of WO 2008/128942, which is incorporated herein by reference in its entirety.

### SUMMARY OF THE INVENTION

The present application relates to novel pharmaceutical compositions comprising crystalline forms of gepotidacin. Gepotidacin has the structure of Formula (I).

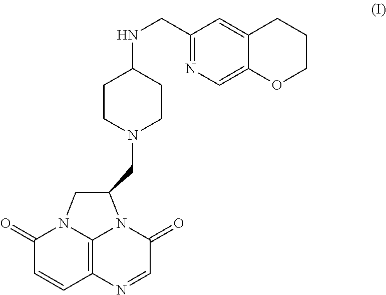 [SMILES: O=c1ccc2ncc(=O)n3c2n1C[C@H]3CN1CCC(NCc2cc3c(cn2)OCCC3)CC1]

The present disclosure provides a novel pharmaceutical compositions comprising gepotidacin, such as crystalline forms of gepotidacin, e.g. gepotidacin mesylate dihydrate, gepotidacin mesylate anhydrate and gepotidacin anhydrate. The present disclosure also provides methods for making the pharmaceutical composition comprising gepotidacin, e.g. crystalline forms of gepotidacin, and methods of treating bacterial infections using the pharmaceutical compositions comprising gepotidacin.

### BRIEF DESCRIPTION OF THE DRAWINGS

FIG. 1 shows an X-ray powder diffraction pattern of gepotidacin mesylate dihydrate (Form 1).

FIG. 2 shows a Raman spectrum of gepotidacin mesylate dihydrate (Form 1).

FIG. 3 shows a differential scanning calorimetry trace of gepotidacin mesylate dihydrate (Form 1).

FIG. 4 shows a thermogravimetric analysis trace of gepotidacin mesylate dihydrate (Form 1).

FIG. 5 shows an X-ray powder diffraction pattern of gepotidacin anhydrate.

FIG. 6 shows a Raman spectrum of gepotidacin anhydrate.

FIG. 7 shows a differential scanning calorimetry trace of gepotidacin anhydrate.

FIG. 8 shows a thermogravimetric analysis trace of gepotidacin anhydrate.

FIG. 9 shows an X-ray powder diffraction pattern of gepotidacin mesylate dihydrate (Form 1).

FIG. 10 shows an X-ray powder diffraction pattern of gepotidacin anhydrate.

FIG. 11 shows a manufacturing process for tablets comprising gepotidacin mesylate dihydrate.

FIG. 12 shows the tabletability profile for Formulation E (circles) and Formulation F (squares).

FIG. 13 shows the comparison of disintegration times as a function of tablet tensile strength for Formulation E (circles) and Formulation F (squares).

FIG. 14 shows an X-ray powder diffraction pattern of gepotidacin mesylate anhydrate.

FIG. 15 shows an X-ray powder diffraction pattern of gepotidacin mesylate anhydrate.

### DETAILED DESCRIPTION OF THE INVENTION

The present application is directed to pharmaceutical compositions comprising gepotidacin. In some embodiments the gepotidacin in the compositions is crystalline gepotidacin, such as gepotidacin mesylate dihydrate (Form 1), gepotidacin mesylate anhydrate or gepotidacin anhydrate (free base). International Patent Application Publication No. WO 2021/219637 describes certain crystalline forms of gepotidacin, including gepotidacin mesylate dihydrate, gepotidacin mesylate anhydrate, gepotidacin mesylate monohydrate and gepotidacin anhydrate (free base).

In some embodiments, the pharmaceutical compositions of the present invention comprise crystalline gepotidacin.

Thus in one embodiment, the present invention provides a pharmaceutical composition comprising a crystalline form of gepotidacin and one or more pharmaceutically acceptable excipients, wherein the crystalline form is gepotidacin mesylate dihydrate, gepotidacin mesylate anhydrate or gepotidacin anhydrate, and wherein the pharmaceutical composition comprises about 45% to 75% by weight of gepotidacin (measured as free base).

In one embodiment, the present invention provides a pharmaceutical composition comprising a crystalline form of gepotidacin and one or more pharmaceutically acceptable excipients, wherein the crystalline form is gepotidacin mesylate dihydrate or gepotidacin anhydrate, and wherein the pharmaceutical composition comprises about 45% to 75% by weight of gepotidacin (measured as free base).

### 1. Gepotidacin Mesylate Dihydrate (Form 1)

In some embodiments, the crystalline form of gepotidacin in the pharmaceutical compositions is gepotidacin mesylate dihydrate (i.e. Form 1). Gepotidacin mesylate dihydrate can be represented by the structure below.

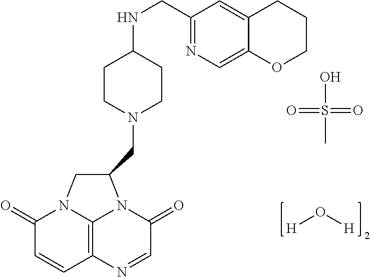

In one embodiment, gepotidacin mesylate dihydrate is characterized by an X-ray powder diffraction (XRPD) pattern comprising at least three, at least four, at least five, at least six, at least seven, at least eight, or at least nine diffraction angles, when measured using Cu K_{α} radiation, selected from the group consisting of about 9.0, 11.5, 13.4, 14.3, 14.9, 15.5, 17.6, 18.6, and 20.7 degrees 2θ. As used herein, when the term “about” is before a list of numbers, the term applies to each of the listed numbers. In one embodiment, gepotidacin mesylate dihydrate is characterized by an XRPD pattern comprising at least three diffraction angles, when measured using Cu K_{α} radiation, selected from the group consisting of about 13.4, 15.5, 17.6, and 18.6 degrees 2θ. In one embodiment, gepotidacin mesylate dihydrate is characterized by an XRPD pattern comprising four diffraction angles, when measured using Cu K_{α} radiation, at about 13.4, 15.5, 17.6, and 18.6 degrees 2θ. In one embodiment, gepotidacin mesylate dihydrate is characterized by an XRPD pattern comprising three diffraction angles, when measured using Cu K_{α} radiation, at about 13.4, 17.6, and 18.6 degrees 2θ.

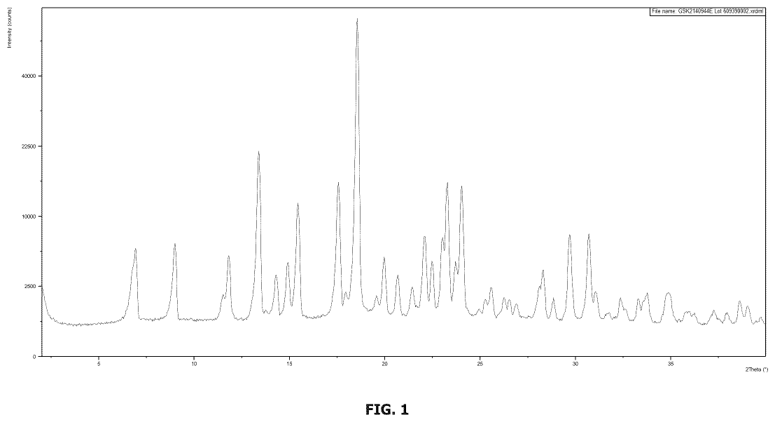

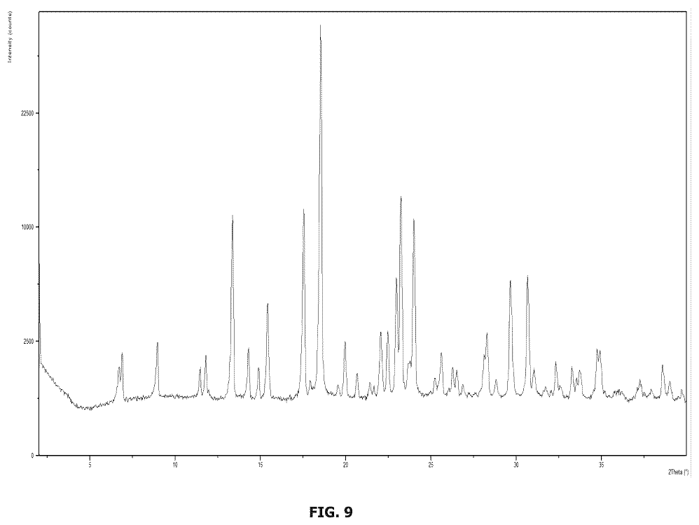

In one embodiment, gepotidacin mesylate dihydrate is characterized by an XRPD pattern substantially in accordance with FIG. 1. In one embodiment, gepotidacin mesylate dihydrate is characterized by an XRPD pattern substantially in accordance with FIG. 9.

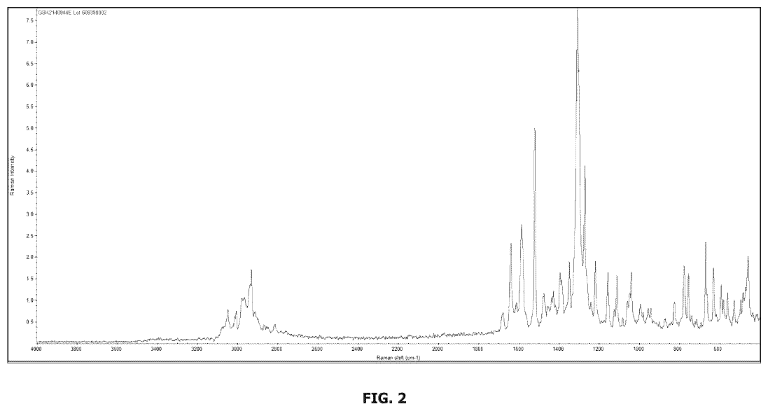

In one embodiment, gepotidacin mesylate dihydrate is characterized by a Raman spectrum comprising at least three, at least four, at least five, at least six, or at least seven peaks at positions selected from the group consisting of peaks at about 1154, 1269, 1306, 1518, 1584, 1637 and 1676 cm⁻¹. In one embodiment, gepotidacin mesylate dihydrate is characterized by a Raman spectrum substantially in accordance with FIG. 2.

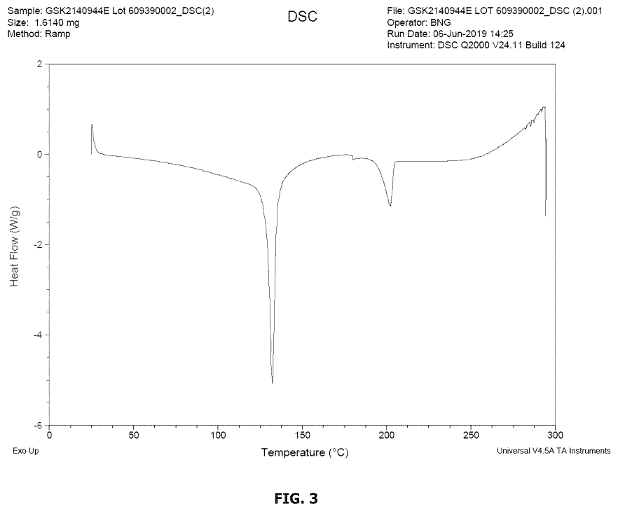

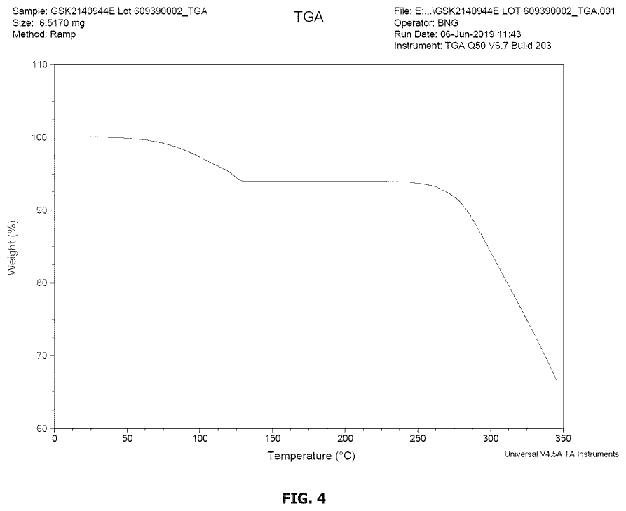

In further embodiments, gepotidacin mesylate dihydrate is characterized by a differential scanning calorimetry trace substantially in accordance with FIG. 3 and/or a thermogravimetric analysis trace substantially in accordance with FIG. 4.

In another embodiment, gepotidacin mesylate dihydrate is characterized by single crystal XRD resulting in the following unit cell parameters:
a=6.9255(5) Å; b=15.4500(12) 521 ; c=25.7918(19) Å; α=β=γ=90°;
V=2759.7(4) Å³; Z′=1;
Space group P2₁2₁2₁;
Molecules/unit cell 4;
Density (calculated) 1.398 g/cm³;
wherein Z′ is the number of molecules per asymmetric unit.

### 2. Gepotidacin Anhydrate

In some embodiments, the crystalline form of gepotidacin is in the pharmaceutical compositions gepotidacin anhydrate (i.e., free base). In one embodiment, gepotidacin anhydrate is characterized by an XRPD pattern comprising at least three, at least four, at least five, at least six, at least seven, at least eight, or at least nine diffraction angles, when measured using Cu Kα radiation, selected from the group consisting of about 8.8, 10.8, 11.7, 12.8, 13.2, 14.4, 16.3, 19.9, 20.8, and 25.0. In one embodiment, gepotidacin anhydrate is characterized by an XRPD pattern comprising at least three diffraction angles, when measured using Cu K_{α} radiation, selected from the group consisting of about 8.8, 13.2, 14.4, and 20.8. In one embodiment, gepotidacin anhydrate is characterized by an XRPD pattern comprising four diffraction angles, when measured using Cu K_{α} radiation, at about 8.8, 13.2, 14.4, and 20.8. In one embodiment, gepotidacin anhydrate is characterized by an XRPD pattern comprising three diffraction angles, when measured using Cu K_{α} radiation, at about 8.8, 13.2, and 14.4.

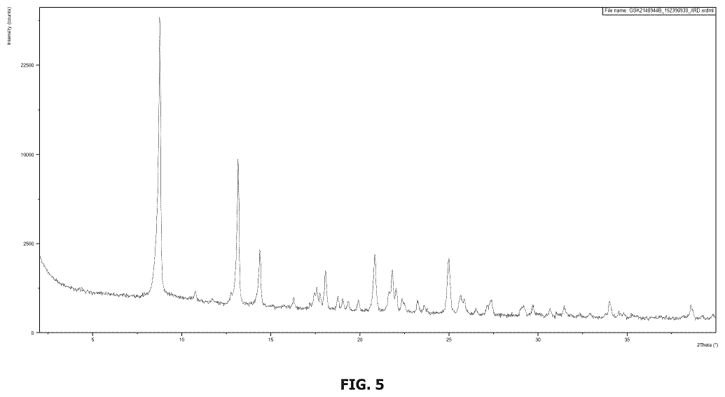

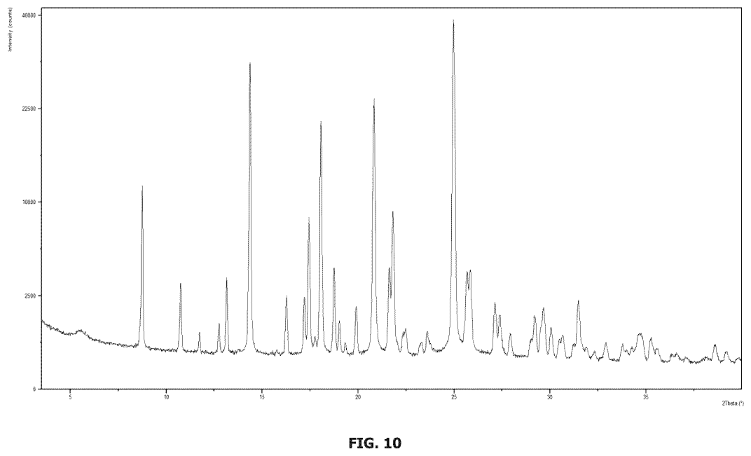

In one embodiment, gepotidacin anhydrate is characterized by an XRPD pattern substantially in accordance with FIG. 5. In one embodiment, gepotidacin anhydrate is characterized by an XRPD pattern substantially in accordance with FIG. 10.

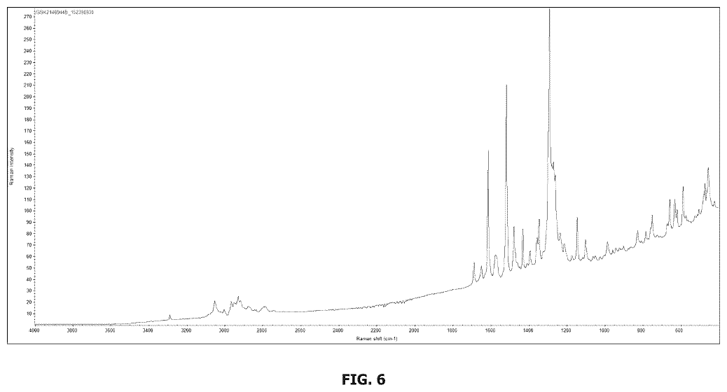

In one embodiment, gepotidacin anhydrate is characterized by a Raman spectrum comprising at least three, at least four, at least five, at least six, at least seven, or at least eight peaks at positions selected from the group consisting of peaks at about 1099, 1143, 1289, 1344, 1476, 1516, 1612, and 1687 cm⁻¹. In one embodiment, gepotidacin anhydrate is characterized by a Raman spectrum substantially in accordance with FIG. 6.

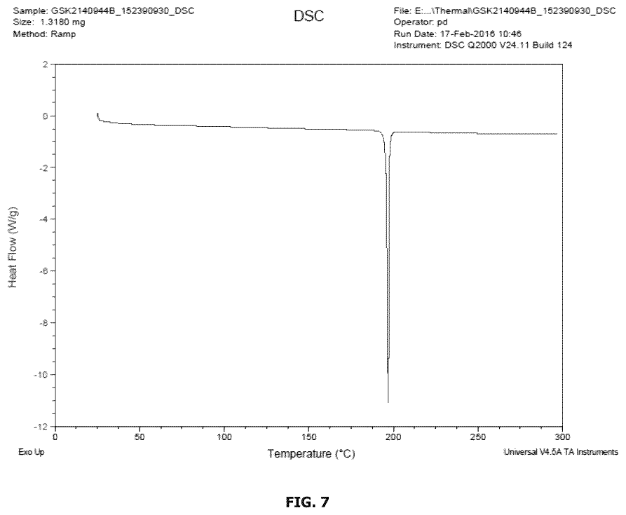

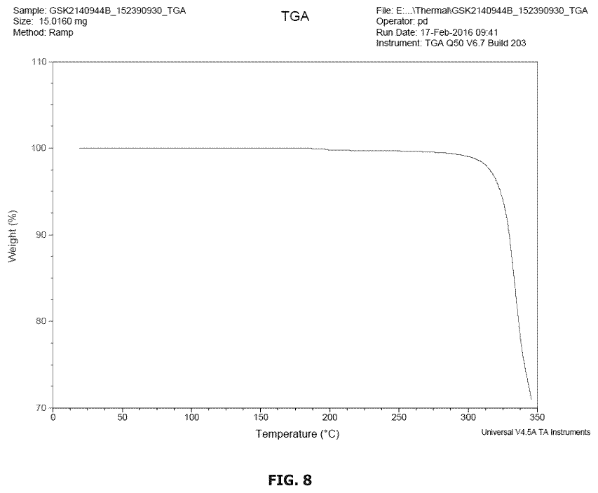

In further embodiments, gepotidacin anhydrate is characterized by a differential scanning calorimetry trace substantially in accordance with FIG. 7 and/or a thermogravimetric analysis trace substantially in accordance with FIG. 8.

In another embodiment, gepotidacin anhydrate is characterized by single crystal XRD resulting in the following unit cell parameters: a=8.44022(16) Å; b=6.42442(12) Å; c=20.2774(5) Å; α=γ=90°; β=96.778(2)°; V=1091.83(4) Å³; Z′=1;
Space group P2₁;
Drug molecules/unit cell 2;
Density (calculated) 1.364 g/cm²;
wherein Z′ is the number of drug molecules per asymmetric unit.

### Gepotidacin Mesylate Anhydrate

In some embodiments, the crystalline form of gepotidacin is gepotidacin mesylate anhydrate. In one embodiment, gepotidacin mesylate anhydrate is characterized by an XRPD pattern comprising at least three, at least four, at least five, at least six, or at least seven diffraction angles, when measured using Cu Kα radiation, selected from the group consisting of about 7.1, 9.7, 12.1, 14.2, 15.2, 17.3, and 20.2. In one embodiment, gepotidacin mesylate anhydrate is characterized by an XRPD pattern comprising at least three diffraction angles, when measured using Cu Kα radiation, selected from the group consisting of about 7.1, 9.7, 15.2, and 17.3. In one embodiment, gepotidacin mesylate anhydrate is characterized by an XRPD pattern comprising four diffraction angles, when measured using Cu Kα radiation, at about 7.1, 9.7, 15.2, and 17.3. In one embodiment, gepotidacin mesylate anhydrate is characterized by an XRPD pattern comprising three diffraction angles, when measured using Cu Kα radiation, at about 9.7, 15.2, and 17.3.

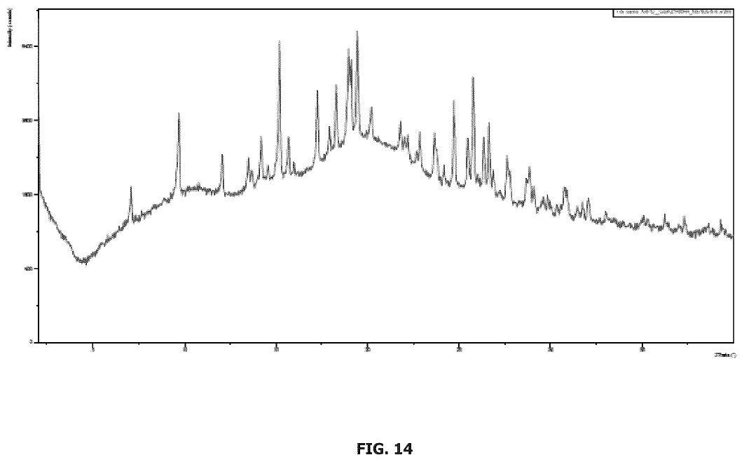

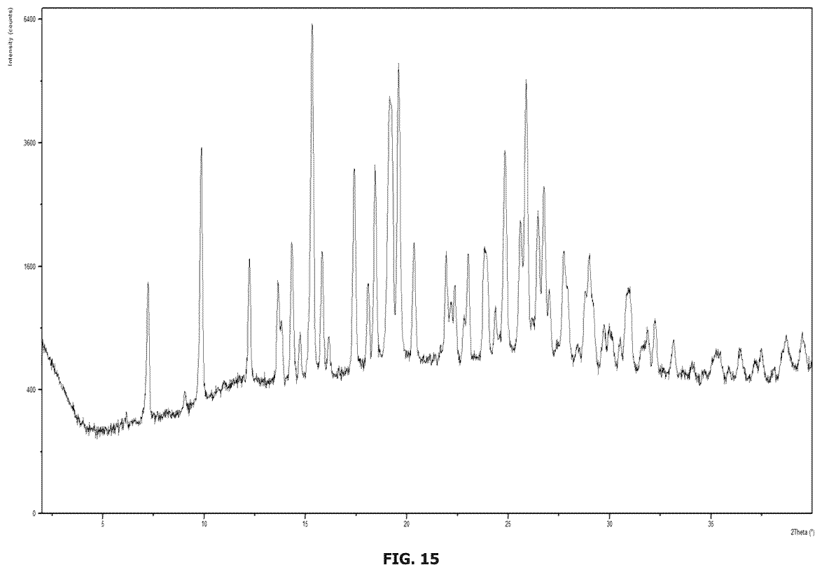

In one embodiment, gepotidacin mesylate anhydrate is characterized by an XRPD pattern substantially in accordance with FIG. 14. In one embodiment, gepotidacin mesylate anhydrate is characterized by an XRPD pattern substantially in accordance with FIG. 15.

In still further embodiments, as a person having ordinary skill in the art will understand, a particular gepotidacin polymorph is characterized by any combination of two or more sets of the analytical data characterizing the aforementioned embodiments. For example, in one embodiment, gepotidacin mesylate dihydrate is characterized by an X-ray powder diffraction (XRPD) pattern substantially in accordance with FIG. 1 or FIG. 9, a Raman spectrum substantially in accordance with FIG. 2, a differential scanning calorimetry trace substantially in accordance with FIG. 3, and a thermogravimetric analysis trace substantially in accordance with FIG. 4.

In another embodiment, gepotidacin mesylate dihydrate is characterized by an X-ray powder diffraction (XRPD) pattern substantially in accordance with FIG. 1 and a Raman spectrum substantially in accordance with FIG. 2. In another embodiment, gepotidacin mesylate dihydrate is characterized by an X-ray powder diffraction (XRPD) pattern substantially in accordance with FIG. 1 and a differential scanning calorimetry trace substantially in accordance with FIG. 3. In another embodiment, gepotidacin mesylate dihydrate is characterized by an X-ray powder diffraction (XRPD) pattern substantially in accordance with FIG. 1 and a thermogravimetric analysis trace substantially in accordance with FIG. 4.

An XRPD pattern will be understood to comprise a diffraction angle (expressed in degrees 2θ) of “about” a value specified herein when the XRPD pattern comprises a diffraction angle within ±0.2 degrees 2θ of the specified value. Further, it is well known and understood to those skilled in the art that the apparatus employed, humidity, temperature, orientation of the powder crystals, and other parameters involved in obtaining an X-ray powder diffraction (XRPD) pattern may cause some variability in the appearance, intensities, and positions of the lines in the diffraction pattern. The term “XRPD” is used herein exchangeably with the term

“PXRD.”

An X-ray powder diffraction pattern that is “substantially in accordance” with that of FIG. 1, 5, 9, or 10 provided herein is an XRPD pattern that would be considered by one skilled in the art to represent a compound possessing the same crystal form as the compound that provided the XRPD pattern of FIG. 1, 5, 9, or 10. That is, the XRPD pattern may be identical to that of FIG. 1, 5, 9, or 10, or more likely it may be somewhat different. Such an XRPD pattern may not necessarily show each of the lines of any one of the diffraction patterns presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns. For example, one skilled in the art can overlay an XRPD pattern of a sample containing gepotidacin, with FIG. 1 and, using expertise and knowledge in the art, readily determine whether the XRPD pattern of the sample is substantially in accordance with the XRPD pattern of gepotidacin mesylate dihydate (Form 1). If the XRPD pattern is substantially in accordance with FIG. 1, the sample form can be readily and accurately identified as having the same form as Form 1.

The present application provides high drug load oral solid formulations comprising gepotidacin. In some embodiments, the present application provides high drug load oral solid formulations comprising a crystalline form of gepotidacin. The proposed clinical oral dose of gepotidacin is 1500 mg twice a day for treating urinary tract infection (UTI) and 3000 mg twice a day for treating an infection by Neisseria gonorrhoeae (e.g. uncomplicated gonorrhea or urogenital gonorrhea). Such high daily doses would require high drug load oral solid formulations. High drug loading means a smaller amount of excipients with a reasonable tablet weight, and would result in more sensitivity of drug product to variation in drug substance over the lifecycle including variation in disintegration/dissolution behavior, compression characteristics, flow behavior, with very few excipients to compensate for the variation in drug substance properties.

In some embodiments, the high load formulation of the present application is a tablet. In some embodiments, the high load formulation of the present application is a capsule. In some embodiments, gepotidacin in the high load tablet formulation is present in an amount of about 45%, 50%, 55%, 60%, 65%, 70%, or 75% by weight (measured as free base), or in a range between any two preceeding values. In some embodiments, gepotidacin is present in a range of about 45% to 75%, about 45% to 70%, about 45% to 65%, about 45% to 60%, about 50% to 75%, about 50% to 70%, about 50% to 65%, about 50% to 60%, or about 50% to 55% by weight. As used herein, when the term “about” is before a list of numbers, the term applies to each of the listed numbers.

Unless otherwise indicated, the weight percentage is calculated by using the weight of the active ingredient (i.e., gepotidacin free base, gepotidacin mesylate anhydrate or gepotidacin mesylate dihydrate) against the weight of the composition (i.e., tablets). For example, for tablets of 1400 mg containing about 971 mg gepotidacin mesylate dihydrate, the weight percentage of gepotidacin mesylate dihydrate is about 69.4% and the weight percentage of gepotidacin (measured as free base) is about 53.6%.

In one aspect, the high load formulation comprises gepotidacin mesylate dihydrate, and gepotidacin mesylate dihydrate is present in an amount of about 60%, 65%, 70%, 75%, 80%, 85%, or 90% by weight, or in a range between any two preceeding values. In some embodiments, gepotidacin mesylate dihydrate is present in a range of about 60% to 90%, about 60% to 80%, about 65% to 85%, about 65% to 75% by weight. In some embodiments, gepotidacin mesylate dihydrate is present in an amount of about 70% by weight.

In another aspect, the high load formulation comprises gepotidacin anhydrate and gepotidacin anhydrate is present in an amount of about 50%, 55%, 60%, 65%, 70%, 75%, 80%, or 85% by weight, or in a range between any two preceeding values. In some embodiments, gepotidacin anhydrate is present in a range of about 50% to 85%, about 50% to 80%, about 50% to 75%, about 50% to 70%, about 50% to 65%, about 55% to 70%, about 55% to 65%, or about 60% to 65% by weight. In some embodiments, gepotidacin anhydrate is present in an amount of about 63% by weight.

In one aspect, the high load formulation comprises gepotidacin mesylate anhydrate, and gepotidacin mesylate dihydrate is in an amount of about 60%, 65%, 70%, 75%, 80%, 85%, or 90% by weight, or in a range between any two preceeding values. In some embodiments, gepotidacin mesylate anhydrate is present in a range of about 60% to 90%, about 60% to 80%, about 65% to 85%, about 65% to 75% by weight. In some embodiments, gepotidacin mesylate anhydrate is present in an amount of about 70% by weight.

In one embodiment, the high load formulation comprises gepotidacin mesylate dihydrate, a diluent, a disintegrant, and a glidant. In another embodiment, the high load formulation comprises gepotidacin mesylate dihydrate, a diluent, a disintegrant, a glidant, and a lubricant.

In one embodiment, the high load formulation comprises gepotidacin anhydrate, a diluent, and a disintegrant. In another embodiment, the high load formulation comprises gepotidacin anhydrate, a diluent, a disintegrant, and a lubricant.

In one embodiment, the high load formulation comprises gepotidacin mesylate anhydrate, a diluent, and a disintegrant. In another embodiment, the high load formulation comprises gepotidacin mesylate anhydrate, a diluent, a disintegrant, and a lubricant.

Suitable diluents (i.e., filler) include lactose, sucrose, dextrose, mannitol, sorbitol, starch (e.g. corn starch, potato starch, and pre-gelatinized starch), cellulose and its derivatives (e.g., microcrystalline cellulose), calcium sulfate, and dibasic calcium phosphate. In one embodiment, the diluent is selected from the group consisting of microcrystalline cellulose, lactose, sucrose, dextrose, mannitol, sorbitol, starch, cellulose, calcium sulfate, dibasic calcium phosphate, and a combination thereof. In one embodiment, the diluent is microcrystalline cellulose. In one embodiment, the diluent is present in an amount of about 20% to 25% by weight of the composition.

Suitable disintegrants include crospovidone, sodium starch glycolate, croscarmellose, croscarmellose sodium, alginic acid, sodium alginate, and sodium carboxymethyl cellulose. In one embodiment, the disintegrant is selected from the group consisting of crospovidone, sodium starch glycolate, croscarmellose, croscarmellose sodium, alginic acid, sodium alginate, sodium carboxymethyl cellulose, and a combination thereof. In one embodiment, the disintegrant is croscarmellose sodium. In one embodiment, the disintegrant is present in an amount of about 4% to 10% by weight of the composition.

Suitable lubricants include stearic acid, magnesium stearate, calcium stearate, sodium stearyl fumarate, and talc. In one embodiment, the lubricant is selected from the group consisting of stearic acid, magnesium stearate, calcium stearate, sodium stearyl fumarate, talc, and a combination thereof. In one embodiment, the lubricant is magnesium stearate. In one embodiment, the lubrican is present in an amount of about 0.5% to 5%, or 0.5% to 2%, by weight of the composition.

Glidants include colloidal silicon dioxide (i.e., silica). Certain lubricants can be used as glidants, such as talc, and magnesium stearate, stearic acid, and sodium stearyl fumarate. In one embodiment, the glidant is selected from the group consisting of colloidal silicon dioxide, magnesium stearate, and a combination thereof. In one embodiment, the glidant is colloidal silicon dioxide. In one embodiment, the glidant is present in an amount of about 0.5% to 2% by weight of the composition.

### 1) Formulations of Gepotidacin Mesylate Dihydrate

In some embodiments, the pharmaceutical composition disclosed herein comprises:
about 65% to 75% by weight of gepotidacin mesylate dihydrate,
about 20% to 25% by weight of the diluent,
about 4% to 10% by weight of the disintegrant, and
about 0.5% to 2% by weight of the glidant.

In one embodiment, the pharmaceutical composition disclosed herein comprises:
about 65% to 75% by weight of gepotidacin mesylate dihydrate,
about 20% to 25% by weight of microcrystalline cellulose,
about 4% to 10% by weight of croscarmellose sodium, and
about 0.5% to 2% by weight of colloidal silicon dioxide.

In some embodiments, the pharmaceutical composition further comprise about 0.5% to 5% by weight of a lubricant, or about 0.5% to 2% by weight of a lubricant, for example, magnesium stearate.

In some embodiments, the pharmaceutical composition comprises:
about 70% by weight of gepotidacin mesylate dihydrate,
about 22% by weight of the diluent,
about 6% by weight of the disintegrant, and
about 1% by weight of the glidant.

In some embodiments, the pharmaceutical composition comprises:
about 70% by weight of gepotidacin mesylate dihydrate,
about 22% by weight of microcrystalline cellulose,
about 6% by weight of croscarmellose sodium, and
about 1% by weight of colloidal silicon dioxide.

In some embodiments, the pharmaceutical composition comprises:
about 70% by weight of gepotidacin mesylate dihydrate,
about 22% by weight of the diluent,
about 6% by weight of the disintegrant, and
about 0.5% by weight of the glidant.

In some embodiments, the pharmaceutical composition comprises:
about 70% by weight of gepotidacin mesylate dihydrate,
about 22% by weight of microcrystalline cellulose,
about 6% by weight of croscarmellose sodium, and
about 0.5% by weight of colloidal silicon dioxide.

In some embodiments, the pharmaceutical composition comprises:
about 70% by weight of gepotidacin mesylate dihydrate,
about 22% by weight of the diluent,
about 6% by weight of the disintegrant,
about 1% by weight of the glidant, and
about 1.4% by weight of the lubricant.

In some embodiments, the pharmaceutical composition comprises:
about 70% by weight of gepotidacin mesylate dihydrate,
about 22% by weight of microcrystalline cellulose,
about 6% by weight of croscarmellose sodium,
about 1% by weight of colloidal silicon dioxide, and
about 1.4% by weight of magnesium stearate.

In some embodiments, the pharmaceutical composition comprises:
about 70% by weight of gepotidacin mesylate dihydrate,
about 22% by weight of the diluent,
about 6% by weight of the disintegrant,
about 0.5% by weight of the glidant, and
about 1.9% by weight of the lubricant.

In some embodiments, the pharmaceutical composition comprises:
about 70% by weight of gepotidacin mesylate dihydrate,
about 22% by weight of microcrystalline cellulose,
about 6% by weight of croscarmellose sodium,
about 0.5% by weight of colloidal silicon dioxide, and
about 1.9% by weight of magnesium stearate.

In some embodiments, the pharmaceutical composition comprises:
about 971 mg of gepotidacin mesylate dihydrate,
about 311 mg of microcrystalline cellulose,
about 84 mg of croscarmellose sodium,
about 14 mg of colloidal silicon dioxide, and
about 19.5 mg of magnesium stearate.

In some embodiments, the pharmaceutical composition comprises:
about 971 mg of gepotidacin mesylate dihydrate,
about 311 mg of microcrystalline cellulose,
about 84 mg of croscarmellose sodium,
about 7 mg of colloidal silicon dioxide, and
about 26.5 mg of magnesium stearate.

In another aspect, the pharmaceutical composition comprises about 60% to 80% by weight of gepotidacin mesylate dihydrate, intragranular excipients, and extragranular excipients. In some embodiments, the intragranular excipients comprise:
about 4% to 8% by weight of a diluent,
about 1% to 5% by weight of a disintegrant, and
about 0.1% to 1% by weight of a glidant.

In some embodiments, the intragranular excipients comprise:
about 4% to 8% by weight of microcrystalline cellulose,
about 1% to 5% by weight of croscarmellose sodium, and
about 0.1% to 1% by weight of colloidal silicon dioxide.

In some embodiments, the extragranular excipients comprise:
about 10% to 20% by weight of a diluent,
about 1% to 5% by weight of a disintegrant, and
about 0.1% to 1% by weight of a glidant.

In some embodiments, the extragranular excipients comprise:
about 10% to 20% by weight of microcrystalline cellulose,
about 1% to 5% by weight of croscarmellose sodium, and
about 0.1% to 1% by weight of colloidal silicon dioxide.

In some embodiments, the extragranular excipients comprise:
about 10% to 20% by weight of a diluent, and
about 1% to 5% by weight of a disintegrant.

In some embodiments, the extragranular excipients comprise:
about 10% to 20% by weight of microcrystalline cellulose, and
about 1% to 5% by weight of croscarmellose sodium.

In some embodiments, the intragranular excipients further comprise about 0.1% to 3% by weight of a lubricant, and the extragranular excipients further comprise about 0.1% to 3% by weight of a lubricant. Each excipient in the intragranular and extragranular excipients can be independently selected. For example, the diluent in the intragranular excipients can be the same or different from the diluent in the extragranular excipients.

In some embodiments, the pharmaceutical composition comprises:
about 65% to 75% by weight of gepotidacin mesylate dihydrate,
the intragranular excipients comprising:
   about 5% to 7% by weight of the diluent,
   about 2% to 4% by weight of the disintegrant,
   about 0.4% to 0.6% by weight of the glidant, and
   about 0.2% to 0.6% by weight of the lubricant, and
the extragranular excipients comprising:
   about 14% to 18% by weight of the diluent,
   about 2% to 4% by weight of the disintegrant,
   about 0.4% to 0.6% by weight of the glidant, and
   about 0.5% to 1.5% by weight of the lubricant.

In some embodiments, the pharmaceutical composition comprises:
about 65% to 75% by weight of gepotidacin mesylate dihydrate,
the intragranular excipients comprising:
   about 5% to 7% by weight of microcrystalline cellulose,
   about 2% to 4% by weight of croscarmellose sodium,
   about 0.4% to 0.6% by weight of colloidal silicon dioxide, and
   about 0.2% to 0.6% by weight of magnesium stearate, and
the extragranular excipients comprising:
   about 14% to 18% by weight of microcrystalline cellulose,
about 2% to 4% by weight of croscarmellose sodium,
about 0.4% to 0.6% by weight of colloidal silicon dioxide, and
about 0.5% to 1.5% by weight of magnesium stearate.

In some embodiments, the pharmaceutical composition comprises:
about 70% by weight of gepotidacin mesylate dihydrate,
the intragranular excipients comprising:
   about 6% by weight of the diluent,
   about 3% by weight of the disintegrant,
   about 0.5% by weight of the glidant, and
   about 0.4% by weight of the lubricant, and
the extragranular excipients comprising:
   about 16% by weight of the diluent,
   about 3% by weight of the disintegrant,
   about 0.5% by weight of the glidant, and
   about 1% by weight of the lubricant.

In some embodiments, the pharmaceutical composition comprises:
about 70% by weight of gepotidacin mesylate dihydrate,
the intragranular excipients comprising:
   about 6% by weight of the diluent,
   about 3% by weight of the disintegrant,
   about 0.5% by weight of the glidant, and
   about 0.4% by weight of the lubricant, and
the extragranular excipients comprising:
   about 16% by weight of the diluent,
   about 3% by weight of the disintegrant, and
   about 1.5% by weight of the lubricant.

In some embodiments, the pharmaceutical composition comprises:
about 70% by weight of gepotidacin mesylate dihydrate,
the intragranular excipients comprising:
   about 6% by weight of microcrystalline cellulose,
   about 3% by weight of croscarmellose sodium,
   about 0.5% by weight of colloidal silicon dioxide, and
   about 0.4% by weight of magnesium stearate, and
the extragranular excipients comprising:
   about 16% by weight of microcrystalline cellulose,
   about 3% by weight of croscarmellose sodium,
   about 0.5% by weight of colloidal silicon dioxide, and
   about 1% by weight of magnesium stearate.

In some embodiments, the pharmaceutical composition comprises:
about 70% by weight of gepotidacin mesylate dihydrate,
the intragranular excipients comprising:
   about 6% by weight of microcrystalline cellulose,
   about 3% by weight of croscarmellose sodium,
   about 0.5% by weight of colloidal silicon dioxide, and
   about 0.4% by weight of magnesium stearate, and
the extragranular excipients comprising:
   about 16% by weight of microcrystalline cellulose,
   about 3% by weight of croscarmellose sodium, and
   about 1.5% by weight of magnesium stearate.

In some embodiments, the pharmaceutical composition comprises:
about 971 mg of gepotidacin mesylate dihydrate,
the intragranular excipients comprising:
   about 84.5 mg of microcrystalline cellulose,
   about 42 mg of croscarmellose sodium,
   about 7 mg of colloidal silicon dioxide, and
   about 5.5 mg of magnesium stearate, and the extragranular excipients comprising:
   about 227 mg of microcrystalline cellulose,
   about 42 mg of croscarmellose sodium,
   about 7 mg of colloidal silicon dioxide, and
   about 14 mg of magnesium stearate.

In some embodiments, the pharmaceutical composition comprises:
about 971 mg of gepotidacin mesylate dihydrate,
the intragranular excipients comprising:
   about 84.5 mg of microcrystalline cellulose,
   about 42 mg of croscarmellose sodium,
   about 7 mg of colloidal silicon dioxide, and
   about 5.5 mg of magnesium stearate, and
the extragranular excipients comprising:
   about 227 mg of microcrystalline cellulose,
   about 42 mg of croscarmellose sodium, and
   about 21 mg of magnesium stearate.

### 2) Formulations of Gepotidacin Anhydrate

In some embodiments, the pharmaceutical composition disclosed herein comprises:
about 60% to 65% by weight of gepotidacin anhydrate,
about 25% to 35% by weight of the diluent, and
about 4% to 8% by weight of the disintegrant.

In some embodiments, the pharmaceutical composition further comprises about 0.5% to 5% by weight of a lubricant.
In some embodiments, the pharmaceutical composition comprises:
   about 63% by weight of gepotidacin anhydrate,
   about 31% by weight of the diluent, and
   about 5% by weight of the disintegrant.
In some embodiments, the pharmaceutical composition comprises:
   about 63% by weight of gepotidacin anhydrate,
   about 31% by weight of the diluent,
   about 5% by weight of the disintegrant, and
   about 1% by weight of the lubricant.
In some embodiments, the pharmaceutical composition comprises:
   about 750 mg of gepotidacin anhydrate,
   about 375 mg of microcrystalline cellulose,
   about 64 mg of croscarmellose sodium, and
   about 11 mg of magnesium stearate.
In some embodiments, the pharmaceutical composition comprises about 55% to 70% by weight of gepotidacin anhydrate, intragranular excipients, and extragranular excipients. In some embodiments, the intragranular excipients comprise:
about 10% to 15% by weight of a diluent, and
about 1% to 5% by weight of a disintegrant,

In some embodiments, the extragranular excipients comprise:
about 15% to 20% by weight of a diluent, and
about 1% to 5% by weight of a disintegrant.

In some embodiments, the intragranular excipients further comprise about 0.1% to 3% by weight of a lubricant, and the extragranular excipients further comprise about 0.1% to 3% by weight of a lubricant.

In some embodiments, the pharmaceutical composition comprises:
about 60% to 65% by weight of gepotidacin anhydrate,
the intragranular excipients comprising:
   about 11% to 15% by weight of the diluent,
   about 1% to 4% by weight of the disintegrant, and
   about 0.2% to 0.6% by weight of the lubricant, and
the extragranular excipients comprising:
   about 16% to 20% by weight of the diluent,
   about 2% to 4% by weight of the disintegrant, and
   about 0.3% to 0.7% by weight of the lubricant.

In some embodiments, the pharmaceutical composition comprises:
about 63% by weight of gepotidacin anhydrate,
the intragranular excipients comprising:
   about 13% by weight of the diluent,
   about 2.3% by weight of the disintegrant, and
   about 0.4% by weight of the lubricant, and
the extragranular excipients comprising:
   about 18% by weight of the diluent,
   about 3% by weight of the disintegrant, and
   about 0.5% by weight of the lubricant.

In some embodiments, the pharmaceutical composition comprises:
about 750 mg of gepotidacin anhydrate,
the intragranular excipients comprising:
   about 155 mg of microcrystalline cellulose,
   about 28 mg of croscarmellose sodium, and
   about 4.7 mg of magnesium stearate, and
the extragranular excipients comprising:
   about 221 mg of microcrystalline cellulose,
   about 36 mg of croscarmellose sodium, and
   about 6 mg of magnesium stearate.

In some embodiments, the pharmaceutical compositions disclosed herein are tablets. In some embodiments, the tablets are coated with a film.

### 3) Formulations of Gepotidacin Mesylate Anhydrate

In some embodiments, the pharmaceutical composition disclosed herein comprises:
about 65% to 75% by weight of gepotidacin mesylate anhydrate,
about 20% to 25% by weight of the diluent,
about 4% to 10% by weight of the disintegrant, and
about 0.5% to 2% by weight of the glidant.

In one embodiment, the pharmaceutical composition disclosed herein comprises:
about 65% to 75% by weight of gepotidacin mesylate anhydrate,
about 20% to 25% by weight of microcrystalline cellulose,
about 4% to 10% by weight of croscarmellose sodium, and
about 0.5% to 2% by weight of colloidal silicon dioxide.

### 3. Use and Method of Treatment

The present application provides a method for treating bacterial infections in a human in need thereof comprising administering to the human a pharmaceutical composition comprising an effective amount of gepotidacin. The bacterial infections can be caused by a wide range of organisms including both Gram-negative and Gram-positive organisms, and the infections include, but are not limited to, upper and/or lower respiratory tract infections, skin and soft tissue infections, urinary tract infections, and gonorrhea. In some embodiments, the infection is urinary tract infection. In some embodiments, the infection is gonorrhoea. The method of treating bacterial infections by using gepotidacin is disclosed in WO2008/128942, WO2016/027249 and WO2020/201833, all of which are incorporated herein by reference in their entirety.

In some embodiments, the infection is urinary tract infection caused by Escherichia coli (E. coli), Staphylococcus saprophyticus, Citrobacter koseri, or Klebsiella pneumoniae (K. pneumoniae). In some embodiments, the infection is urinary tract infection caused by E. coli. In another embodiment, the infection is gonorrhoea caused by Neisseria gonorrhoeae.

As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician. Unless otherwise stated, the amount of a drug or pharmaceutical agent refers to the amount of the free base compound, not the amount of the corresponding pharmaceutically acceptable salt.

In one embodiment, the present application provides a method for treating urinary tract infection (UTI), comprising administering the pharmaceutical composition disclosed herein, in a therapeutically effective amount in a human subject in need thereof, wherein the gepotidacin is administered at 1500 mg twice a day, 6-12 hours apart.

In particular, a pharmaceutical composition of the present application is presented as a unit dose and taken preferably from 1 to 5 times daily, such as once or twice daily to achieve the desired effect. In one embodiment, gepotidacin is administered for any of 3, 4, 5, 6 or 7 continuous days. In one embodiment, in any aspect of the present application, gepotidacin is administered for 5 continuous days.

In another embodiment, the present application provides a method for treating an infection by Neisseria gonorrhoeae, comprising administering the pharmaceutical composition disclosed herein in a therapeutically effective amount in a human subject in need thereof, wherein the gepotidacin is administered twice, each at 3000 mg, 6-12 hours apart.

In another embodiment, the present application provides a method for treating gonorrhea, comprising administering the pharmaceutical composition disclosed herein in a therapeutically effective amount in a human subject in need thereof, wherein the gepotidacin is administered twice, each at 3000 mg, 6-12 hours apart.

The invention is illustrated by the following clauses:
1. A pharmaceutical composition comprising gepotidacin and one or more pharmaceutically acceptable excipients, wherein the pharmaceutical composition comprises about 45% to 75% by weight of gepotidacin (measured as free base).
1a. A pharmaceutical composition comprising a crystalline form of gepotidacin and one or more pharmaceutically acceptable excipients, wherein the crystalline form is gepotidacin mesylate dihydrate or gepotidacin anhydrate, and wherein the pharmaceutical composition comprises about 45% to 75% by weight of gepotidacin (measured as free base).
2. The pharmaceutical composition according to clause 1, wherein the gepotidacin is in a crystalline form.
3. The pharmaceutical composition according to clause 2, wherein the crystalline form is gepotidacin mesylate dihydrate, gepotidacin mesylate anhydrate or gepotidacin anhydrate.
4. The pharmaceutical composition according to clause 1a or 3, wherein the crystalline form is gepotidacin mesylate dihydrate.
5. The pharmaceutical composition according to clause 4, wherein the gepotidacin mesylate dihydrate is characterized by an X-ray powder diffraction (XRPD) pattern comprising at least three or at least four diffraction angles, when measured using Cu K_{α} radiation, selected from the group consisting of about 9.0, 11.5, 13.4, 14.3, 14.9, 15.5, 17.6, 18.6, and 20.7 degrees 2θ.
6. The pharmaceutical composition according to clause 4, wherein the gepotidacin mesylate dihydrate is characterized by an X-ray powder diffraction (XRPD) pattern substantially in accordance with FIG. 1.
7. The pharmaceutical composition according to any one of clauses 4 to 6, wherein the pharmaceutical composition comprises about 60% to 90% by weight of gepotidacin mesylate dihydrate.
8. The pharmaceutical composition according to any one of clauses 4 to 7, wherein the pharmaceutical composition comprises about 60% to 80% by weight of gepotidacin mesylate dihydrate, and wherein the one or more excipients comprise a diluent, a disintegrant, and a glidant.
9. The pharmaceutical composition according to clause 8, comprising:
about 65% to 75% by weight of gepotidacin mesylate dihydrate,
about 20% to 25% by weight of the diluent,
about 4% to 10% by weight of the disintegrant, and
about 0.5% to 2% by weight of the glidant.
10. The pharmaceutical composition according to clause 8 or 9, wherein
the diluent is selected from the group consisting of microcrystalline cellulose, lactose, sucrose, dextrose, mannitol, sorbitol, starch, cellulose, calcium sulfate, dibasic calcium phosphate, and a combination thereof;
the disintegrant is selected from the group consisting of crospovidone, sodium starch glycolate, croscarmellose, croscarmellose sodium, alginic acid, sodium alginate, sodium carboxymethyl cellulose, and a combination thereof; and
the glidant is selected from the group consisting of colloidal silicon dioxide, magnesium stearate, and a combination thereof.
11. The pharmaceutical composition according to any one of clauses 7 to 10, wherein the one or more excipients further comprise a lubricant.
12. The pharmaceutical composition according to clause 11, wherein the lubricant is selected from the group consisting of stearic acid, magnesium stearate, calcium stearate, sodium stearyl fumarate, talc, and a combination thereof.
13. The pharmaceutical composition according to clause 11 or 12, wherein the lubricant comprises about 0.5% to 5% by weight.
13. The pharmaceutical composition according to clause 9, comprising:
about 70% by weight of gepotidacin mesylate dihydrate,
about 22% by weight of the diluent,
about 6% by weight of the disintegrant, and
about 1% by weight of the glidant.
14. The pharmaceutical composition according to clause 9, comprising:
about 70% by weight of gepotidacin mesylate dihydrate,
about 22% by weight of the diluent,
about 6% by weight of the disintegrant,
about 1% by weight of the glidant, and
about 1.4% by weight of the lubricant.
15. The pharmaceutical composition according to clause 9, comprising:
about 70% by weight of gepotidacin mesylate dihydrate,
about 22% by weight of the diluent,
about 6% by weight of the disintegrant,
about 0.5% by weight of the glidant, and
about 1.9% by weight of the lubricant.
16. The pharmaceutical composition according to any one of clauses 4-7 wherein the pharmaceutical composition comprises about 60% to 80% by weight of gepotidacin mesylate dihydrate, intragranular excipients, and extragranular excipients,
wherein the intragranular excipients comprise
   about 4% to 8% by weight of a diluent,
   about 1% to 5% by weight of a disintegrant, and
   about 0.1% to 1% by weight of a glidant, and
the extragranular excipients comprise
about 10% to 20% by weight of a diluent, and
about 1% to 5% by weight of a disintegrant.
17. The pharmaceutical composition according to clause 16, wherein the extragranular excipients further comprise about 0.1% to 1% by weight of a glidant.
18. The pharmaceutical composition according to clause 16 clause 17, wherein
each said diluent is independently selected from the group consisting of microcrystalline cellulose, lactose, sucrose, dextrose, mannitol, sorbitol, starch, cellulose, calcium sulfate, dibasic calcium phosphate, and a combination thereof;
each said disintegrant is independently selected from the group consisting of crospovidone, sodium starch glycolate, croscarmellose, croscarmellose sodium, alginic acid, sodium alginate, sodium carboxymethyl cellulose, and a combination thereof; and
each said glidant is independently selected from the group consisting of colloidal silicon dioxide, magnesium stearate, and a combination thereof.
19. The pharmaceutical composition according to any one of clauses 16 to 18, wherein the intragranular excipients further comprise about 0.1% to 3% by weight of a lubricant, and the extragranular excipients further comprise about 0.1% to 3% by weight of a lubricant.
20. The pharmaceutical composition according to clause 19, wherein each said lubricant is independently selected from the group consisting of stearic acid, magnesium stearate, calcium stearate, sodium stearyl fumarate, talc, and a combination thereof.
21. The pharmaceutical composition according to any one of clauses 16 to 20, comprising:
about 65% to 75% by weight of gepotidacin mesylate dihydrate,
the intragranular excipients comprising:
   about 5% to 7% by weight of the diluent,
   about 2% to 4% by weight of the disintegrant,
   about 0.4% to 0.6% by weight of the glidant, and
   about 0.2% to 0.6% by weight of the lubricant, and
the extragranular excipients comprising:
   about 14% to 18% by weight of the diluent,
   about 2% to 4% by weight of the disintegrant,
   about 0.4% to 0.6% by weight of the glidant, and
   about 0.5% to 1.5% by weight of the lubricant.
22. The pharmaceutical composition according to clause 21, comprising:
about 70% by weight of gepotidacin mesylate dihydrate,
the intragranular excipients comprising:
   about 6% by weight of the diluent,
   about 3% by weight of the disintegrant,
   about 0.5% by weight of the glidant, and
   about 0.4% by weight of the lubricant, and
the extragranular excipients comprising:
   about 16% by weight of the diluent,
   about 3% by weight of the disintegrant,
   about 0.5% by weight of the glidant, and
   about 1% by weight of the lubricant.
23. The pharmaceutical composition according to clause 1a or 3, wherein the crystalline form is gepotidacin anhydrate.
24. The pharmaceutical composition according to clause 23, wherein the gepotidacin anhydrate is characterized by an X-ray powder diffraction (XRPD) pattern comprising at least three or at least four diffraction angles, when measured using Cu K_{α} radiation, selected from the group consisting of about 8.8, 10.8, 11.7, 12.8, 13.2, 14.4, 16.3, 19.9, 20.8, and 25.0 degrees 2θ.
25. The pharmaceutical composition according to clause 23, wherein the gepotidacin anhydrate characterized by an X-ray powder diffraction (XRPD) pattern substantially in accordance with FIG. 4.
26. The pharmaceutical composition according to any one of clauses 23 to 25, wherein the pharmaceutical composition comprises about 50% to 75% by weight of gepotidacin anhydrate.
27. The pharmaceutical composition according to any one of clauses 23 to 26, wherein the pharmaceutical composition comprises about 55% to 70% by weight of gepotidacin anhydrate, and wherein the one or more excipients comprise a diluent and a disintegrant.
28. The pharmaceutical composition according to clause 26, wherein the one or more excipients further comprise a lubricant.
29. The pharmaceutical composition according to clause 27, wherein
the diluent is selected from the group consisting of microcrystalline cellulose, lactose, sucrose, dextrose, mannitol, sorbitol, starch, cellulose, calcium sulfate, dibasic calcium phosphate, and a combination thereof; and
the disintegrant is selected from the group consisting of crospovidone, sodium starch glycolate, croscarmellose, croscarmellose sodium, alginic acid, sodium alginate, sodium carboxymethyl cellulose, and a combination thereof.
30. The pharmaceutical composition according to clause 23 to 29, wherein the one or more excipients further comprise a lubricant selected from the group consisting of stearic acid, magnesium stearate, calcium stearate, sodium stearyl fumarate, talc, and a combination thereof.
31. The pharmaceutical composition according to any one of clauses 27 to 30, comprising:
about 60% to 65% by weight of gepotidacin anhydrate,
about 25% to 35% by weight of the diluent, and
about 4% to 8% by weight of the disintegrant.
32. The pharmaceutical composition according to any one of clauses 27 to 31, the one or more excipients further comprise about 0.5% to 5% by weight of a lubricant.
33. The pharmaceutical composition according to clause 31, comprising:
about 63% by weight of gepotidacin anhydrate,
about 31% by weight of the diluent, and
about 5% by weight of the disintegrant.
34. The pharmaceutical composition according to clause 31, comprising:
about 63% by weight of gepotidacin anhydrate,
about 31% by weight of the diluent,
about 5% by weight of the disintegrant, and
about 1% by weight of the lubricant.
35. The pharmaceutical composition according to clause 27, comprising:
about 750 mg of gepotidacin anhydrate,
about 375 mg of microcrystalline cellulose,
about 64 mg of croscarmellose sodium, and
about 11 mg of magnesium stearate.
36. The pharmaceutical composition according to any one of clauses 26 to 30, wherein the pharmaceutical composition comprises about 55% to 70% by weight of gepotidacin anhydrate, intragranular excipients, and extragranular excipients,
wherein the intragranular excipients comprise
   about 10% to 15% by weight of a diluent, and
   about 1% to 5% by weight of a disintegrant, and
the extragranular excipients comprise
about 15% to 20% by weight of a diluent, and
about 1% to 5% by weight of a disintegrant.
37. The pharmaceutical composition according to clause 36, wherein
each said diluent is selected from the group consisting of microcrystalline cellulose, lactose, sucrose, dextrose, mannitol, sorbitol, starch, cellulose, calcium sulfate, dibasic calcium phosphate, and a combination thereof; and
each said disintegrant is selected from the group consisting of crospovidone, sodium starch glycolate, croscarmellose, croscarmellose sodium, alginic acid, sodium alginate, sodium carboxymethyl cellulose, and a combination thereof.
38. The pharmaceutical composition according to clause 36 or 37, wherein the intragranular excipients further comprise about 0.1% to 3% by weight of a lubricant, and the extragranular excipients further comprise about 0.1% to 3% by weight of a lubricant.
39. The pharmaceutical composition according to clause 38, wherein each said lubricant is independently selected from the group consisting of stearic acid, magnesium stearate, calcium stearate, sodium stearyl fumarate, talc, and a combination thereof.
40. The pharmaceutical composition according to any one of clauses 36 to 39, comprising:
about 60% to 65% by weight of gepotidacin anhydrate,
the intragranular excipients comprising:
   about 11% to 15% by weight of the diluent,
   about 1% to 4% by weight of the disintegrant, and
   about 0.2% to 0.6% by weight of the lubricant, and
the extragranular excipients comprising:
   about 16% to 20% by weight of the diluent,
   about 2% to 4% by weight of the disintegrant, and
   about 0.3% to 0.7% by weight of the lubricant.
41. The pharmaceutical composition according to clause 40, comprising:
about 63% by weight of gepotidacin anhydrate,
the intragranular excipients comprising:
   about 13% by weight of the diluent,
   about 2.3% by weight of the disintegrant, and
   about 0.4% by weight of the lubricant, and
the extragranular excipients comprising:
   about 18% by weight of the diluent,
   about 3% by weight of the disintegrant, and
   about 0.5% by weight of the lubricant.
42. The pharmaceutical composition according to clause 36, comprising:
about 750 mg of gepotidacin anhydrate,
the intragranular excipients comprising:
   about 155 mg of microcrystalline cellulose,
   about 28 mg of croscarmellose sodium, and
   about 4.7 mg of magnesium stearate, and
the extragranular excipients comprising:
   about 221 mg of microcrystalline cellulose,
   about 36 mg of croscarmellose sodium, and
   about 6 mg of magnesium stearate.
43. The pharmaceutical composition according to any one of clauses 1 to 42, which is in the form of a tablet.
44. The pharmaceutical composition according to clause 43, wherein the tablet is coated with a film.
45. A method of treating a bacterial infection in a human in need thereof comprising administering to the human the pharmaceutical composition according to any one of clauses 1 to 44.
46. The method according to clause 45, wherein the bacterial infection is uncomplicated urinary tract infection or an infection by Neisseria gonorrhoeae.
47. The method according to clause 45, wherein the bacterial infection is uncomplicated urinary tract infection, and the pharmaceutical composition is administered at a dose of 1500 mg of gepotidacin (measured as free base) twice daily for 5 days.
48. The method according to clause 45, wherein the bacterial infection is infection by Neisseria gonorrhoeae, and the pharmaceutical composition is administered at a dose of 3000 mg of gepotidacin (measured as free base), followed by a second dose of 3000 mg after 10-12 hours.
49. The pharmaceutical composition according to any one of clauses 1 to 42 for use in therapy.
50. The pharmaceutical composition according to any of clauses 1 to 42 for use in treating uncomplicated urinary tract infection or an infection by Neisseria gonorrhoeae.

The Examples set forth below are illustrative of the present invention and are not intended to limit, in any way, the scope of the present invention.

### EXPERIMENTALS

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention. Unless otherwise noted, reagents are commercially available or are prepared according to procedures in the literature.

### EXAMPLE 1: Gepotidacin Mesylate Dihydrate (Form 1)

### Example 1a—Preparation Method 1

Acetone (5 ml) was added to gepotidacin (294.14 mg). To the slurry, methanesulfonic acid (3M solution in water, 1 equivalent) was added over a period of 60 minutes. The slurry was heated to 50° C. for 3 hours, cooled slowly to 20° C., left stirring at 20° C. for 5 hours and cooled further to 5° C. The slurry was stirred at 5° C. overnight. The crystalline solids were filtered under vacuum, washed with acetone and dried in a vacuum oven at 60° C. to give crystalline gepotidacin mesylate dihydrate (Form 1) in 72.9% yield.

### Example 1b—Preparation Method 2

Gepotidacin (32.00 kg) and methanesulfonic acid (7.00 kg, 1.02 eq) were heated to 74-80° C. in 304 L of 2-propanol and 16.1 kg water. The solution was filtered into a crystallisation vessel and cooled to 59-63° C. Form 1 dihydrate (0.318 kg) suspended in 5% v/v aqueous 2-propanol (1.194 kg 2-propanol and 0.080 L water) was added and the mixture aged at 58-64° C. for 2 hours. The mixture was cooled to 15-25° C. and the resulting slurry was wet-milled. The slurry was heated to 55-61° C. and cooled to 15-25° C. Gepotidacin mesylate dihydrate was isolated by filtration, washed twice with 5% v/v aqueous 2-propanol (2×106 L 2-propanol and 2×5.6 kg water), and dried under vacuum at about 40° C. to give gepotidacin mesylate dihydrate (Form 1) (38.505 kg) as a crystalline solid.

### Example 1c—XRPD

The X-ray powder diffraction (XRPD) pattern of gepotidacin mesylate dihydrate (Form 1) is shown in FIG. 1 and a summary of the diffraction angle and d-spacings is given in table 1 below. The XRPD analysis was conducted on a PANalytical X'Pert Pro diffractometer on Si zero-background wafers. The acquisition conditions included Cu K_{α} radiation, generator tension 45 kV, generator current: 40 mA, step size 0.03°2θ.

TABLE 1

| Diff Angle [°2θ] | d-spacing [Å] |
|---|---|
| 6.9 | 12.7 |
| 9.0 | 9.8 |
| 11.5 | 7.7 |
| 11.8 | 7.5 |
| 13.4 | 6.6 |
| 14.3 | 6.2 |
| 14.9 | 5.9 |
| 15.5 | 5.7 |
| 17.6 | 5.0 |
| 17.9 | 4.9 |
| 18.6 | 4.8 |
| 19.6 | 4.5 |
| 20.0 | 4.4 |
| 20.7 | 4.3 |
| 21.4 | 4.1 |
| 22.1 | 4.0 |
| 22.5 | 4.0 |
| 23.0 | 3.9 |
| 23.3 | 3.8 |
| 23.7 | 3.8 |
| 24.0 | 3.7 |
| 24.9 | 3.6 |
| 25.3 | 3.5 |
| 25.6 | 3.5 |
| 26.2 | 3.4 |
| 26.5 | 3.4 |
| 26.9 | 3.3 |
| 28.1 | 3.2 |
| 28.3 | 3.2 |
| 28.8 | 3.1 |
| 29.7 | 3.0 |
| 30.7 | 2.9 |
| 31.1 | 2.9 |
| 31.7 | 2.8 |
| 32.3 | 2.8 |
| 32.6 | 2.7 |
| 33.3 | 2.7 |
| 33.8 | 2.7 |
| 34.7 | 2.6 |
| 35.0 | 2.6 |
| 35.8 | 2.5 |
| 36.2 | 2.5 |
| 37.3 | 2.4 |
| 37.9 | 2.4 |
| 38.6 | 2.3 |
| 39.0 | 2.3 |
| 39.7 | 2.3 |

The XRPD patterns of another sample of gepotidacin mesylate dihydrate (Form 1) is shown in FIG. 9 (see Table 2). The XRPD analysis was conducted on a PANalytical Empyreen difractometer on Si zero-background wafers. The acquisition conditions included Cu K_{α} radiation, generator tension 45 kV, generator current: 40 mA, step size 0.03°2θ.

TABLE 2

| Diff Angle (2θ°) | d-spacing [Å] |
|---|---|
| 6.7 | 13.2 |
| 6.9 | 12.8 |
| 9.0 | 9.9 |
| 11.5 | 7.7 |
| 11.8 | 7.5 |
| 13.4 | 6.6 |
| 14.3 | 6.2 |
| 14.9 | 5.9 |
| 15.4 | 5.7 |
| 17.5 | 5.1 |
| 17.9 | 5.0 |
| 18.5 | 4.8 |
| 19.6 | 4.5 |
| 20.0 | 4.4 |
| 20.7 | 4.3 |
| 21.4 | 4.1 |
| 21.7 | 4.1 |
| 22.1 | 4.0 |
| 22.5 | 4.0 |
| 23.0 | 3.9 |
| 23.2 | 3.8 |
| 23.7 | 3.8 |
| 24.0 | 3.7 |
| 25.2 | 3.5 |
| 25.6 | 3.5 |
| 26.3 | 3.4 |
| 26.5 | 3.4 |
| 26.9 | 3.3 |
| 28.1 | 3.2 |
| 28.3 | 3.2 |
| 28.8 | 3.1 |
| 29.7 | 3.0 |
| 30.7 | 2.9 |
| 31.0 | 2.9 |
| 31.7 | 2.8 |
| 32.3 | 2.8 |
| 33.3 | 2.7 |
| 33.5 | 2.7 |
| 33.7 | 2.7 |
| 34.7 | 2.6 |
| 34.9 | 2.6 |
| 35.9 | 2.5 |
| 37.3 | 2.4 |
| 37.9 | 2.4 |
| 38.6 | 2.3 |
| 39.0 | 2.3 |
| 39.7 | 2.3 |

### Example 1d—Raman Spectrum

The Raman spectrum of gepotidacin mesylate dihydrate (Form 1) was recorded on a Nicolet NXR9650 or Thermo Electron NXR 960 spectrometer, at 4 cm⁻¹ resolution with excitation from a Nd:YVO4 laser (λ=1064 nm). The Raman spectrum of gepotidacin mesylate dihydrate (Form 1) is shown in FIG. 2 with major peaks observed at 455, 492, 558, 525, 590, 628, 667, 752, 775, 823, 940, 993, 1037, 1109, 1154, 1216, 1269, 1306, 1346, 1392, 1424, 1472, 1518, 1584, 1637, 1676, 2929, 3005 and 3046 cm⁻¹.

### Example 1e—DSC

The DSC of gepotidacin mesylate dihydrate (Form 1) was conducted with a TA Instruments Q2000 differential scanning calorimeter equipped with an autosampler and a refrigerated cooling system under 40 mL/min N₂ purge. DSC thermograms of the sample were obtained at 15° C./min in crimped Al pan. The DSC thermogram of Form 1 exhibits a broad endotherm followed by a sharp endotherm with an onset temperature of about 129° C., followed by an endotherm with an onset temperature of about 195° C. (FIG. 3). A person skilled in the art would recognize that the onset temperature of the endotherm may vary depending on the experimental conditions.

### Example 1f—TGA

The thermogravimetric analysis (TGA) thermogram of gepotidacin mesylate dihydrate (Form 1) was recorded on a TA Instruments Q50 thermogravimetric analyzer under 60 mL/min N₂ flow and a heating rate of 10° C./min. The TGA thermogram of gepotidacin mesylate dihydrate (Form 1) exhibits a loss of about 6% (2.0 eq) from 30-130° C. (FIG. 4).

### Example 1g—Single Crystal Structure

A single crystal of gepotidacin mesylate dihydrate was prepared by slow cooling from a solution of gepotidacin mesylate in water/2-propanol.

Single crystal data were collected on a Bruker D8 Venture system using an Incoatec microfocus 3.0 CuKα Source. Data collection and unit cell Indexing were performed in the APEX3 v2017.3-0 suite (Bruker AXS Inc., 2017); processing of the measured intensity data was carried out with the SAINT V8.38A software package (Bruker AXS Inc., 2017). The structures were solved by direct methods using the SHELXT-2018/2 software package (Sheldrick, 2018). The derived atomic parameters (coordinates and temperature factors) were refined through full matrix least-squares in SHELXL-2018/3 (Sheldrick, 2018). Hydrogens were introduced in idealized positions, except for those on heteroatoms, which were freely refined. Single crystal X-ray data were measured at low temperature (−123° C.). The single crystal was confirmed as a mesylate dihydrate structure with the following unit cell parameters: a=6.9255(5) Å; b=15.4500(12) Å; c 32 25.7918(19) Å; α=β=γ=90°; V=2759.7(4) Å³; Z′=1;
Space group P2₁2₁2₁;
Molecules/unit cell 4;
Density (calculated) 1.398 g/cm³;
wherein Z′ is the number of molecules per asymmetric unit.

### Example 1h—Solubility

The solubility of gepotidacin mesylate dihydrate (Form 1) was determined in simulated gastric fluid pH 1.6 (SGF), fasted state simulated intestinal fluid pH 6.5 (FaSSIF) and fed state simulated intestinal fluid pH 6.5 (FeSSIF) at ambient room temperature (20-25 C). See Table 3 below.

TABLE 3

| | Solubility (mg/mL of |
|---|---|
| Media | free base at 20-25° C.) |
| Water | >180 |
| SGF (pH 1.6) | 175 |
| FaSSIF (pH 6.5) | 113 |
| FeSSIF (pH 6.5) | 99 |

### EXAMPLE 2: Gepotidacin Anhydrate

### Example 2a—Preparation Method 1

Gepotidacin (52 g) and 1-propanol (440 mL) was heated to 90° C. 40 mL 1-propanol was added to the clear solution and the combined contents were re-heated to 90° C. The clear solution was cooled to 76° C. and held stirring for 1 hour. The slurry was cooled to 0° C. and held stirring overnight. The slurry was filtered, washed with chilled 1-propanol and dried under vacuum for approximately 6 hours at 50° C. to give gepotidacin anhydrate as a crystalline solid (47.8 g).

### Example 2b—Preparation Method 2

The preparation of gepotidacin anhydrate was carried out on scale according to the following processes:

Charge n-Propanol (12 vol.) to gepotidacin (1.0 equiv) and heat the mixture to 95±3° C. to attain complete dissolution. Filter the mass at 95±3° C. and wash the filters with n-Propanol (0.1 vol). Take filtrate and heat again to 95+3° C. and to ensure complete dissolution. Cool the mass to 77±2° C. Charge seed slurry (1.0% w/w suspended in 2.5 vol n-propanol) and stir for at least 1 h at 77±2° C. Further cool the slurry mass to 0±2° C. and stir for 1 h. Filter the material and wash the cake with n-Propanol (2 vol). Dry the material under vacuum at 50±2° C.

### Example 2c—XRPD

The X-ray powder diffraction (XRPD) pattern of gepotidacin anhydrate is shown in in FIG. 5 and a summary of the diffraction angle and d-spacings is given in table 4 below. The XRPD analysis was conducted on a PANalytical X'Pert Pro diffractometer on Si zero-background wafers. The acquisition conditions included Cu K_{α} radiation, generator tension 45 kV, generator current: 40 mA, step size 0.02°2θ.

TABLE 4

| Diff. Angle [°2θ] | d-spacing [Å] |
|---|---|
| 8.8 | 10.1 |
| 10.8 | 8.2 |
| 11.7 | 7.6 |
| 13.2 | 6.7 |
| 14.4 | 6.2 |
| 16.3 | 5.4 |
| 17.4 | 5.1 |
| 17.6 | 5.0 |
| 17.7 | 5.0 |
| 18.1 | 4.9 |
| 18.7 | 4.7 |
| 19.0 | 4.7 |
| 19.4 | 4.6 |
| 19.9 | 4.5 |
| 20.8 | 4.3 |
| 21.6 | 4.1 |
| 21.8 | 4.1 |
| 22.0 | 4.0 |
| 22.4 | 4.0 |
| 23.2 | 3.8 |
| 23.6 | 3.8 |
| 25.0 | 3.6 |
| 25.6 | 3.5 |
| 25.9 | 3.4 |
| 26.5 | 3.4 |
| 27.1 | 3.3 |
| 27.4 | 3.3 |
| 29.2 | 3.1 |
| 29.7 | 3.0 |
| 30.7 | 2.9 |
| 31.5 | 2.8 |
| 32.9 | 2.7 |
| 34.0 | 2.6 |
| 38.6 | 2.3 |

The XRPD patterns of another sample of gepotidacin anhydrate is shown in FIG. 10 (see Table 5). The XRPD analysis was conducted on a PANalytical X'Pert Pro diffractometer on Si zero-background wafers. The acquisition conditions included Cu K_{α} radiation, generator tension 45 kV, generator current: 40 mA, step size 0.02°2θ.

TABLE 5

| Diff Angle (2θ°) | d-spacing [Å] |
|---|---|
| 5.5 | 16.1 |
| 8.8 | 10.1 |
| 10.8 | 8.2 |
| 11.7 | 7.5 |
| 12.8 | 6.9 |
| 13.2 | 6.7 |
| 14.4 | 6.2 |
| 15.8 | 5.6 |
| 16.3 | 5.5 |
| 17.2 | 5.2 |
| 17.4 | 5.1 |
| 17.7 | 5.0 |
| 18.1 | 4.9 |
| 18.7 | 4.7 |
| 19.0 | 4.7 |
| 19.3 | 4.6 |
| 19.9 | 4.5 |
| 20.8 | 4.3 |
| 21.6 | 4.1 |
| 21.8 | 4.1 |
| 22.3 | 4.0 |
| 22.5 | 4.0 |
| 23.3 | 3.8 |
| 23.6 | 3.8 |
| 25.0 | 3.6 |
| 25.7 | 3.5 |
| 25.9 | 3.4 |
| 27.1 | 3.3 |
| 27.4 | 3.3 |
| 27.9 | 3.2 |
| 29.2 | 3.1 |
| 29.5 | 3.0 |
| 29.7 | 3.0 |
| 30.1 | 3.0 |
| 30.5 | 2.9 |
| 30.6 | 2.9 |
| 31.5 | 2.8 |
| 31.9 | 2.8 |
| 32.4 | 2.8 |
| 32.9 | 2.7 |
| 33.8 | 2.7 |
| 34.3 | 2.6 |
| 34.6 | 2.6 |
| 34.8 | 2.6 |
| 35.3 | 2.5 |
| 35.6 | 2.5 |
| 36.3 | 2.5 |
| 36.6 | 2.5 |
| 37.1 | 2.4 |
| 38.1 | 2.4 |
| 38.6 | 2.3 |
| 39.2 | 2.3 |

### Example 2d—Raman Spectrum

The Raman spectrum of gepotidacin anhydrate was recorded on a Nicolet NXR9650 or Thermo Electron NXR 960 spectrometer, at 4 cm⁻¹ resolution with excitation from a Nd:YVO4 laser (λ=1064 nm). The Raman spectrum of this material is shown in FIG. 6 with major peaks observed at 453, 471, 586, 630, 656, 748, 825, 985, 1099, 1143, 1289, 1344, 1391, 1429, 1476, 1516, 1572, 1612, 1647, 1687, 2927 and 3051 cm⁻¹.

### Example 2e—DSC

The DSC of gepotidacin anhydrate was conducted with a TA Instruments Q2000 differential scanning calorimeter equipped with an autosampler and a refrigerated cooling system under 40 mL/min N₂ purge. DSC thermograms of samples were obtained at 10° C./min in crimped Al pan. The DSC thermogram of gepotidacin anhydrate exhibits a single endotherm with an onset temperature of about 196° C. (FIG. 7). A person skilled in the art would recognize that the onset temperature of the endotherm may vary depending on the experimental conditions.

### Example 2f—TGA

The thermogravimetric analysis (TGA) thermogram of gepotidacin anhydrate was recorded on a TA Instruments Q50 thermogravimetric analyzer under 25 ml/min N₂ flow and a heating rate of 10° C./min. The TGA thermogram of Anhydrate exhibits a loss of about 0.25% from 25-200° C. (FIG. 8).

### Example 2g—Single Crystal Structure

A single crystal of gepotidacin anhydrate was prepared by seeded slow cooling from a 1-propanol solution.

Single crystal data were collected on an Oxford Diffraction Xcalibur A Nova system using a Nova X-ray CuKα Source. Data collection and unit cell Indexing were performed in the CrysAlisPro 1.171.37.34i suite (Agilent Technologies, 2014); processing of the measured intensity data was also carried out with the CrysAlisPro 1.171.37.34i (Agilent Technologies, 2014) software package. The structures were solved by direct methods using the SHELXT-2018/2 (Sheldrick, 2018) software package. The derived atomic parameters (coordinates and temperature factors) were refined through full matrix least-squares in SHELXL-2018/3 (Sheldrick, 2018). Hydrogens were introduced in idealized positions, except for those on heteroatoms, which were freely refined.

Single crystal X-ray data were measured at low temperature (−123° C.). The single crystal was confirmed as a free base anhydrate structure with the following unit cell parameters: a=8.44022(16) Å; b=6.42442(12) Å; c=20.2774(5) Å; α=γ=90°; β=96.778(2)°; V=1091.83(4) Å3; Z′=1;
Space group P2₁;
Drug molecules/unit cell 2;
Density (calculated) 1.364 g/cm³;
wherein Z′ is the number of drug molecules per asymmetric unit.

Gepotidacin anhydrate exhibits low to moderate solubility (<20 mg/mL) in common solvents and water, except in dichloromethane and trifluoroethanol (>100 mg/mL).

### EXAMPLE 3: Capsule Formulations of Gepotidacin Mesylate Salt

Capsules of gepotidacin mesylate dihydrate (containing 100 mg and 500 mg of gepotidacin free base) were prepared according to Table 6 and used in first time in human (FTIH) clinical trials and Phase 2 clinical trials.

TABLE 6
Capsule Formulation of Gepotidacin Mesylate Salt

| | Capsules Strength | | |
|---|---|---|---|
| | Theoretical Quantity | | |
| | (mg/capsule) | | |
| | | 500 mg | |
| Component | 100 mg | (Formulation A) | Function |
| Gepotidacin mesylate dihydrate¹ | 121.4 | 606.8 | Active |
| Microcrystalline cellulose | 15.7 | 78.5 | Filler/Diluent |
| Magnesium stearate | 1.4 | 5.2 | Lubricant |
| Hard Gelatin Capsule, Size 4, | 1 ea | — | Component |
| Swedish Orange | | | |
| Hard Gelatin Capsule, Size 00, Pink | — | 1 ea | Component |
| Total capsule fill weight (mg) | 138.5 | 690.5 | — |

¹Amount equivalent to deliver 100 mg or 500 mg of gepotidacin free base.

Blended gepotidacin mesylate dihydrate was dry granulated by roller compaction and then granules are encapsulated in size 4 (100 mg) and size 00 (500 mg) capsules. Although GI irritation was identified in phase 1 single dose oral study, this was mitigated with food intake with no impact on exposure. Capsules were stable for 24 months at all storage conditions.

### EXAMPLE 4: Tablet Formulation of Gepotidacin Mesylate Salt

An oral relative bioavailability study (RBA Study 1) was conducted to compare a tablet formulation of gepotidacin mesylate dihydrate (Formulation B), containing 750 mg gepotidacin (measured as free base), presented in Table 7 to the reference Formulation A (500 mg capsules) presented in Table 6. Both the reference capsule formulation and the test tablet formulation utilized gepotidacin mesylate dihydrate and were manufactured using roller compaction granulation process. Gepotidacin mesylate dihydrate was found to have poor flow property and the composition of Formulation B was initially developed to provide a formulation process with reasonable flow performance.

At a single dose of 1500 mg the bioavailability of Formulation B (2×750 mg) was found comparable to that of Formulation A (3×500 mg). And both the tablet and reference capsule provided rapid absorption of gepotidacin.

TABLE 7
Composition of Tablet Formulation used in Oral Relative
Bioavailability Study 1 (Formulation B)

| | Quantity | |
|---|---|---|
| Component | (mg/tablet) | Function |
| Intragranular | | |
| Gepotidacin mesylate | 970.96 | Active |
| dihydrate¹ | | |
| Microcrystalline cellulose | 91.54 | Diluent |
| Croscarmellose sodium | 42.00 | Disintegrant |
| Magnesium stearate | 5.50 | Lubricant |
| Extragranular | | |
| Microcrystalline cellulose | 241.00 | Diluent |
| Croscarmellose sodium | 42.00 | Disintegrant |
| Magnesium stearate | 7.00 | Lubricant |
| Core Tablet Weight | 1400.00 | — |
| Film Coat | | |
| Film coat Beige | 42.00 | Film Coat |
| Purified water² | q.s. | |
| Film coated tablet weight | 1442.00 | |

¹Amount equivalent to deliver 750 mg of gepotidacin free base.
²Removed during processing.

### EXAMPLE 5: Tablet Formulations of Gepotidacin Free Base

A second oral relative bioavailability study (RBA Study 2) was conducted to compare two formulations of gepotidacin tablets, 750 mg, presented in Table 8, to the reference

Formulation A presented in Table 6. Both tablet formulations utilized gepotidacin free base (gepotidacin anhydrate). Formulation C was manufactured using roller compaction (RC) process and Formulation D was manufactured using a high shear wet granulation (HSWG) process.

TABLE 8
Composition of Tablet Formulations of Gepotidacin Free Base

| | Quantity | | |
|---|---|---|---|
| | (mg/tablet)/(wt %) | | |
| Component | Formulation C | Formulation D | Function |
| Intragranular | | | |
| Gepotidacin free base | 750.0/62.5% | 750.0/84.8% | Active |
| Microcrystalline | 154.7/12.9% | — | Filler/ |
| cellulose | | | Diluent |
| Hypromellose | — | 24.0/2.7% | Binder |
| Croscarmellose sodium | 28.1/2.3% | 24.0/2.7% | Disintegrant |
| Magnesium stearate | 4.7/0.4% | — | Lubricant |
| Purified water¹ | — | q.s. | Vehicle |
| Intragranular Sub-total | 937.5/78.1% | 798.0/90.2% | |
| Extragranular | | | |
| Microcrystalline | 220.5/18.4% | 51.3/5.8% | Filler/ |
| cellulose | | | Diluent |
| Croscarmellose sodium | 36.0/3% | 26.5/3% | Disintegrant |
| Magnesium stearate | 6.0/0.5% | 8.9/1% | Lubricant |
| Total Core Tablet | 1200.0/100% | 884.7/100% | — |
| Weight | | | |
| Film Coating | | | |
| Opadry ® II, White | 48.0 | 35.4 | Film Coat |
| 85F48011 | | | |
| Purified water¹ | q.s. | q.s. | Vehicle |
| Total Tablet Weight | 1248.0 | 920.1 | |

¹Removed during processing.

Gepotidacin free base blends with AVICEL PH101 (microcrystalline cellulose), AVICEL PH105 (microcrystalline cellulose) or lactose were prepared to compare the compressibility. The two blends containing microcrystalline cellulose compressed better than the blend containing lactose.

Tablets prepared by HSWG can reduce pill burden or tablets size by increasing drug loading. These tablets met the manufacturability criteria. The relative bioavailability data were collected to evaluate 2 different tablet formulations (Formulation C and Formulation D). The free base RC and HSWG tablets were assessed against the reference mesylate salt capsule (Formulation A). The AUC and the Cmax of the RC tablets and the AUC of the HSWG tablets were determined to be comparable to those of the reference capsules; however the Cmax of the HSWG tablets was determined to be higher than that of the reference capsules.

Although the solubility of gepotidacin anhydrate is lower than that of gepotidacin mesylate dihydrate, gepotidacin anhydrate was found acceptable from solubility perspective for development as also shown by the results from RBA study 2. However, during formulation development, tablets of Formulation C prepared from certain batches of gepotidacin free base drug substance were found to have disintegration issues, which may be attributable to the synthetic route of those batches. Thus, to improve formulation robustness, new formulations for gepotidacin were further studied.

### EXAMPLE 6: Improved Tablet Formulations of Gepotidacin Mesylate Salt

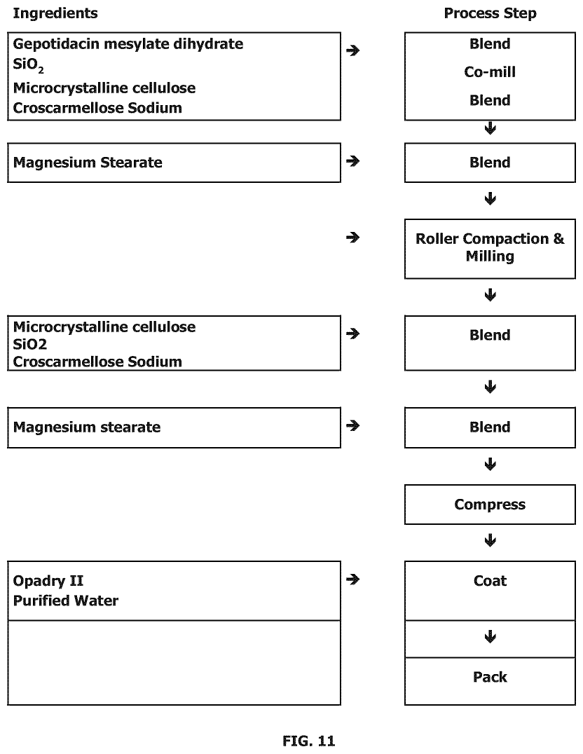

As discussed above, gepotidacin mesylate dihydrate was found to have poor flow property. Studies were conducted to further improve flow performance of the tableting process for gepotidacin mesylate dihydrate. For example, addition of a glidant (e.g. colloidal silicon dioxide) to the intragranular blend was found to help improve flow property. Tablets of Formulation E (see Table 9) were prepared by the process as shown in FIG. 11.

TABLE 9
Composition of Tablet Formulations of Gepotidacin Mesylate Salt

| | Quantity (mg/tablet)/(wt %) | | | |
|---|---|---|---|---|
| Component | Formulation B | Formulation E | Formulation F | Function |
| Intragranular | | | | |
| Gepotidacin | 970.96/69.4% | 970.96/69.4% | 970.96/69.4% | Active |
| mesylate dihydrate¹ | | | | |
| Microcrystalline | 91.54/6.5% | 84.54/6.0% | 84.54/6.0% | Diluent |
| cellulose | | | | |
| Colloidal silicon | — | 7.00/0.5% | 7.00/0.5% | Glidant |
| dioxide | | | | |
| Croscarmellose | 42.00/3% | 42.00/3% | 42.00/3% | Disintegrant |
| sodium | | | | |
| Magnesium | 5.50/0.4% | 5.50/0.4% | 5.50/0.4% | Lubricant |
| stearate | | | | |
| Extragranular | | | | |
| Microcrystalline | 241.00/17.2% | 227.00/16.2% | 227.00/16.2% | Diluent |
| cellulose | | | | |
| Colloidal silicon | — | 7.00/0.5% | — | Glidant |
| dioxide | | | | |
| Croscarmellose | 42.00/3% | 42.00/3% | 42.00/3% | Disintegrant |
| sodium | | | | |
| Magnesium | 7.00/0.5% | 14.00/1% | 21.00/1.5% | Lubricant |
| stearate | | | | |
| Core Tablet Weight | 1400.00/100% | 1400.00/100% | 1400.00/100% | — |
| Film Coat | | | | |
| Film coat Beige | 42.00 | — | — | Film Coat |
| Purified water² | q.s. | | | |
| Film coat Yellow | | 35.00 | 35.00 | Film Coat |
| Purified water² | | q.s. | q.s. | |
| Film coated tablet | 1442.00 | 1435.00 | 1435.00 | |
| weight | | | | |

¹Amount equivalent to deliver 750 mg of gepotidacin free base.
²Removed during processing.

Another tablet formulation with additional magnesium stearate in the extra-granular blend (Table 9, Formulation F) was prepared. Tableting process for Formulation F was observed to have similar flow property as compared to that of Formulation E.

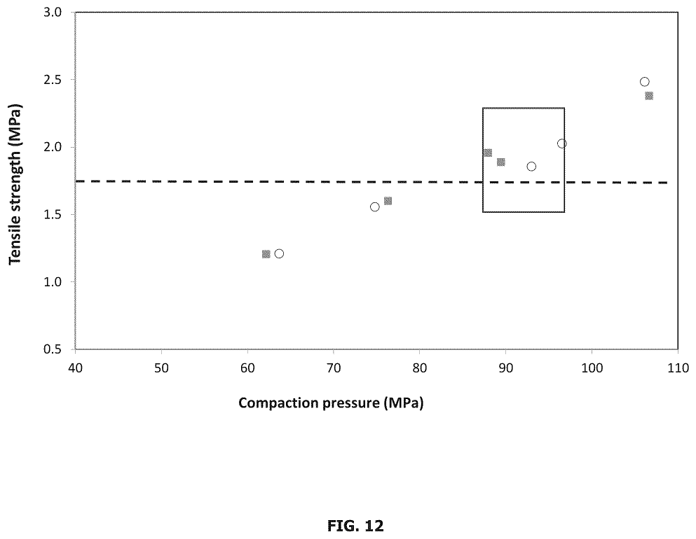

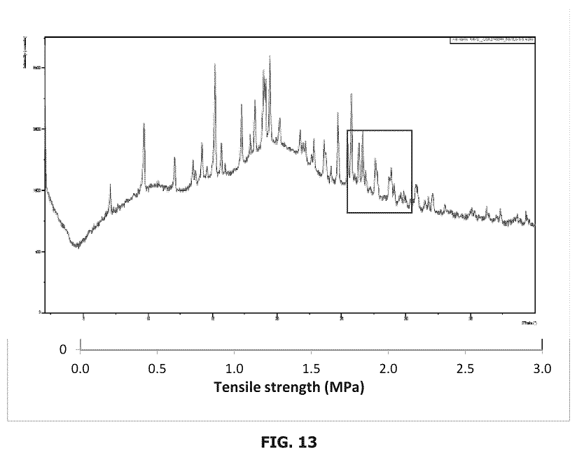

The data in FIG. 12 shows the tabletability profile for Formulation E (circles) and Formulation F (squares). The data shows that: (a) both formulations result in acceptable tabletability profiles, and (b) additional magnesium stearate does not impact tabletability profile. The additional magnesium stearate up to 1.5% w/w level has no impact on tablet disintegration and dissolution either. For example, FIG. 13 shows the comparison of disintegration times as a function of tablet tensile strength for Formulation E (circles) and Formulation F (squares).

An adult and adolescent study was conducted to evaluate the oral gepotidacin mesylate salt 750 mg tablet (Formulation E) for gepotidacin. The data from the single 1,500 mg dose and the two 3,000 mg doses given 6 or 12 h apart confirmed the dose selections in the ongoing Phase 3 studies in acute uncomplicated urinary tract infection (gepotidacin 1,500 mg twice daily for 5 days; NCT04020341 and NCT04187144) and urogenital gonorrhea (gepotidacin two 3,000 mg doses 10 to 12 h apart; NCT04010539), respectively.

Plasma and urine pharmacokinetics data for the gepotidacin mesylate salt capsules and tablets (Formulation A and Formulation B), the two gepotidacin free base tablets (Formulation C and Formulation D), and the improved gepotidacin mesylate salt tablets (Formulation E) are collected and analyzed. Overall, similar systemic plasma and urine exposures were observed after administration of various tablet and capsule formulations of gepotidacin.

### EXAMPLE 7: Gepotidacin Mesylate Anhydrate

### Example 7a—Preparation

Gepotidacin mesylate dihydrate (Form 1) (894 mg) was suspended in isopropyl alcohol (IPA) (5.4 ml) and heated to 61° C. The resultant solids were analysed in-situ by Raman and as a damp slurry by XRPD.

In another preparation, a spatula (<20 mg) of gepotidacin mesylate dihydrate (Form 1) was suspended in IPA (<1.5 ml). The suspension was heated with a heat gun to dissolve most of the solids, and then left to slowly cool to room temperature. The resultant crystals were filtered and analysed by DSC, TGA, and XRPD. Note that this form is unstable under ambient conditions and therefore the resultant analysis may not be for a phase pure anhydrate sample.

### Example 7b—Single Crystal Structure

A single crystal of gepotidacin mesylate anhydrate was prepared by slow cooling from a 2-propanol solution.

Single crystal data were collected on a Bruker D8 Venture system using an Incoatec microfocus 3.0 CuKα Source. Data collection and unit cell Indexing were performed in the APEX3 v2017.3-0 suite (Bruker AXS Inc., 2017); processing of the measured intensity data was carried out with the SAINT V8.38A (Bruker AXS Inc., 2017) software package. The structures were solved by direct methods using the SHELXT-2018/2 (Sheldrick, 2018) software package. The derived atomic parameters (coordinates and temperature factors) were refined through full matrix least-squares in SHELXL-2018/3 (Sheldrick, 2018). Hydrogens were introduced in idealized positions, except for those on heteroatoms, which were freely refined.

Single crystal X-ray data were measured at low temperature (−123° C.). The single crystal was confirmed as a mesylate anhydrate structure with the following unit cell parameters: a=12.3921(7) Å; b=7.0262(4) Å; c=14.6536(9) Å; α=γ=90°; β=95.0077(13)°; V=1271.01(13) Å³; Z′=1;
Space group P2₁;
Drug molecules/unit cell 2;
Density (calculated) 1.423 g/cm³;
wherein Z′ is the number of drug molecules per asymmetric unit.

It is to be understood that the invention is not limited to the aspects or embodiments illustrated hereinabove and the right is reserved to the illustrated aspects or embodiments and all modifications coming within the scope of the following claims.

The various references to journals, patents, and other publications which are cited herein comprise the state of the art and are incorporated herein by reference as though fully set forth.

1. A pharmaceutical composition comprising gepotidacin and one or more pharmaceutically acceptable excipients, wherein the pharmaceutical composition comprises about 45% to 75% by weight of gepotidacin (measured as free base).

2. The pharmaceutical composition according to claim 1, wherein the gepotidacin is in a crystalline form.

3. The pharmaceutical composition according to claim 2, wherein the crystalline form is gepotidacin mesylate dihydrate, gepotidacin mesylate anhydrate or gepotidacin anhydrate.

4. The pharmaceutical composition according to claim 3, wherein the crystalline form is gepotidacin mesylate dihydrate.

5. The pharmaceutical composition according to claim 4, wherein the gepotidacin mesylate dihydrate is characterized by an X-ray powder diffraction (XRPD) pattern comprising at least three or at least four diffraction angles, when measured using Cu K_{α} radiation, selected from the group consisting of about 9.0, 11.5, 13.4, 14.3, 14.9, 15.5, 17.6, 18.6, and 20.7 degrees 2θ.

6. The pharmaceutical composition according to claim 4, wherein the gepotidacin mesylate dihydrate is characterized by an X-ray powder diffraction (XRPD) pattern substantially in accordance with FIG. 1.

7. The pharmaceutical composition according to claim 4, wherein the pharmaceutical composition comprises about 60% to 90% by weight of gepotidacin mesylate dihydrate.

8. The pharmaceutical composition according to claim 4, wherein the pharmaceutical composition comprises about 60% to 80% by weight of gepotidacin mesylate dihydrate, and wherein the one or more excipients comprise a diluent, a disintegrant, and a glidant.

9. The pharmaceutical composition according to claim 8, comprising:
about 65% to 75% by weight of gepotidacin mesylate dihydrate,
about 20% to 25% by weight of the diluent,
about 4% to 10% by weight of the disintegrant, and
about 0.5% to 2% by weight of the glidant.

10. The pharmaceutical composition according to claim 8, wherein
the diluent is selected from the group consisting of microcrystalline cellulose, lactose, sucrose, dextrose, mannitol, sorbitol, starch, cellulose, calcium sulfate, dibasic calcium phosphate, and a combination thereof:
the disintegrant is selected from the group consisting of crospovidone, sodium starch glycolate, croscarmellose, croscarmellose sodium, alginic acid, sodium alginate, sodium carboxymethyl cellulose, and a combination thereof; and
the glidant is selected from the group consisting of colloidal silicon dioxide, magnesium stearate, and a combination thereof.

11. The pharmaceutical composition according to claim 7, wherein the one or more excipients further comprise a lubricant.

12. The pharmaceutical composition according to claim 11, wherein the lubricant is selected from the group consisting of stearic acid, magnesium stearate, calcium stearate, sodium stearyl fumarate, talc, and a combination thereof.

13. The pharmaceutical composition according to claim 11, wherein the lubricant comprises about 0.5% to 5% by weight.

14. The pharmaceutical composition according to claim 3, wherein the crystalline form is gepotidacin anhydrate.

15. The pharmaceutical composition according to claim 14, wherein the gepotidacin anhydrate is characterized by an X-ray powder diffraction (XRPD) pattern comprising at least three or at least four diffraction angles, when measured using Cu K_{α} radiation, selected from the group consisting of about 8.8, 10.8, 11.7, 12.8, 13.2, 14.4, 16.3, 19.9, 20.8, and 25.0 degrees 2θ.

16. The pharmaceutical composition according to claim 14, wherein the gepotidacin anhydrate is characterized by an X-ray powder diffraction (XRPD) pattern substantially in accordance with FIG. 4.

17. The pharmaceutical composition according to claim 1, which is in the form of a tablet.

18. A method of treating a bacterial infection in a human in need thereof comprising administering to the human the pharmaceutical composition according to claim 1.

19. The method according to claim 18, wherein the bacterial infection is uncomplicated urinary tract infection or an infection by Neisseria gonorrhoeae.

20. The method according to claim 18, wherein the bacterial infection is uncomplicated urinary tract infection, and the pharmaceutical composition is administered at a dose of 1500 mg of gepotidacin (measured as free base) twice daily for 5 days.

21. The method according to claim 18, wherein the bacterial infection is infection by Neisseria gonorrhoeae, and the pharmaceutical composition is administered at a dose of 3000 mg of gepotidacin (measured as free base), followed by a second dose of 3000 mg after 10-12 hours.

22-23. (canceled)
